(19) United States
(12) Patent Application Publication
(10) Pub. No.: US 2024/0148196 A1
(43) Pub. Date: May 9, 2024

(54) PERSONAL HYGIENE DEVICE FOR CLEANING A BELLY BUTTON

(71) Applicant: Henry C Flores MD. LLC, Healdsburg, CA (US)

(72) Inventor: Henry C. Flores, Healdsburg, CA (US)

(21) Appl. No.: 18/376,248

(22) Filed: Oct. 3, 2023

Related U.S. Application Data
(60) Provisional application No. 63/413,544, filed on Oct. 5, 2022.

(51) Int. Cl. A47K 7/04

(52) U.S. Cl. CPC A47K 7/043

(57) ABSTRACT

In various embodiments, a personal hygiene device for cleaning a belly button includes a tapered sponge and a handle coupled to and extending from a rear surface of the tapered sponge.

### CROSS REFERENCE TO OTHER APPLICATIONS

This application claims priority to U.S. Provisional Patent Application No. 63/413,544 entitled PERSONAL HYGIENE DEVICE FOR CLEANING A BELLY BUTTON filed Oct. 5, 2022 which is incorporated herein by reference for all purposes.

### BACKGROUND OF THE INVENTION

The belly button tends to harbor dirt, grime, and bacteria, and it is often not sufficiently cleaned. This can be problematic, particularly before surgery or other medical procedures. Before surgery, the body is cleaned with wipes, and a skin preparation is used for cleaning the body. However, the belly button is often missed or not adequately cleaned, leading to an increased risk of infection.

Currently, there are no devices specifically designed for cleaning the belly button. Rather, a belly button may be cleaned with a cotton swab, but cleaning with such a product does not always remove all matter, debris, and bacteria.

### BRIEF DESCRIPTION OF THE DRAWINGS

Various embodiments of the invention are disclosed in the following detailed description and the accompanying drawings.

FIG. 1 shows an example of a personal hygiene device for cleaning a belly button.

FIG. 2 shows an example of a personal hygiene device including a cleaning solution for cleaning a belly button.

### DETAILED DESCRIPTION

The invention can be implemented in numerous ways, including as a process; an apparatus; a system; a composition of matter; a computer program product embodied on a computer readable storage medium; and/or a processor, such as a processor configured to execute instructions stored on and/or provided by a memory coupled to the processor. In this specification, these implementations, or any other form that the invention may take, may be referred to as techniques. In general, the order of the steps of disclosed processes may be altered within the scope of the invention. Unless stated otherwise, a component such as a processor or a memory described as being configured to perform a task may be implemented as a general component that is temporarily configured to perform the task at a given time or a specific component that is manufactured to perform the task. As used herein, the term ‘processor’ refers to one or more devices, circuits, and/or processing cores configured to process data, such as computer program instructions.

A detailed description of one or more embodiments of the invention is provided below along with accompanying figures that illustrate the principles of the invention. The invention is described in connection with such embodiments, but the invention is not limited to any embodiment. The scope of the invention is limited only by the claims and the invention encompasses numerous alternatives, modifications, and equivalents. Numerous specific details are set forth in the following description in order to provide a thorough understanding of the invention. These details are provided for the purpose of example and the invention may be practiced according to the claims without some or all of these specific details. For the purpose of clarity, technical material that is known in the technical fields related to the invention has not been described in detail so that the invention is not unnecessarily obscured.

The embodiments described herein relate generally to personal hygiene devices and, more particularly, to a disposable personal hygiene device for cleaning the belly button. Some embodiments of the present disclosure include a personal hygiene device for cleaning the belly button may include a conical sponge, such as a conical sponge with an auger-like shape, and a handle extending from a rear surface of the conical-sponge. The device may further include a cleaning solution releasably encapsulated within the handle.

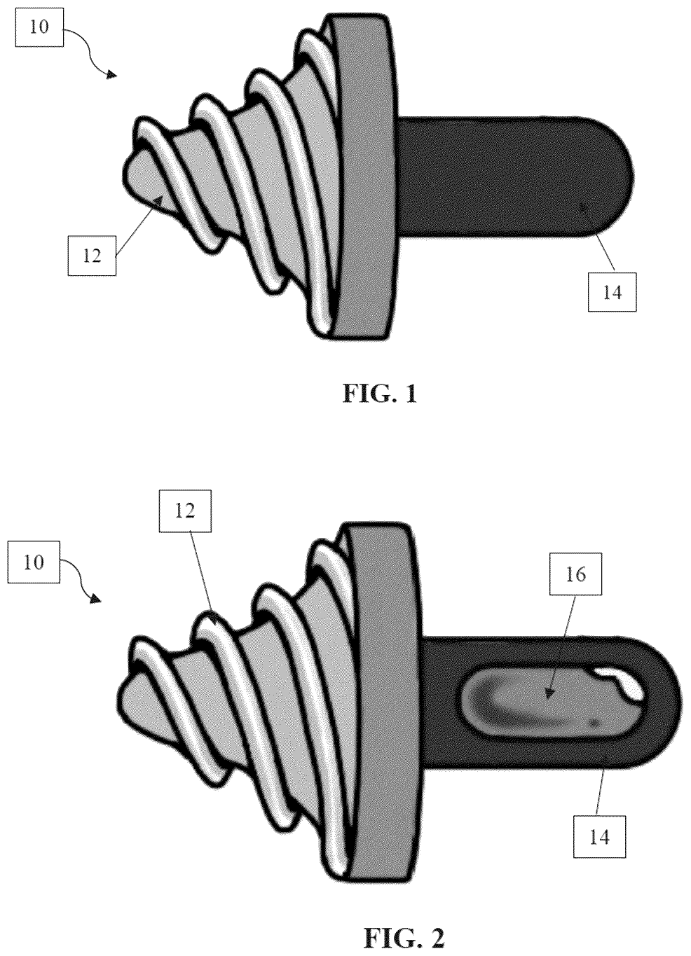

FIG. 1 shows an example of a personal hygiene device for cleaning a belly button. In various embodiments, a personal hygiene device 10 for cleaning the belly button comprises a tapered/conical sponge 12, such as a conical sponge with an auger-like shape, and a handle 14 extending from a rear surface of the conical-sponge 12. In this example, the sponge has a tapering shape that is wider at a base and becomes narrower at the top. This shape may be attractive for cleaning a belly button because it can easily be inserted into a cavity and fill gaps/spaces to thoroughly clean the belly button. In various embodiments, the sponge includes helical grooves as shown. This may help clean the belly button by reaching hard to reach areas of a cavity or recess, e.g., when the device is operated by twisting the handle to cause the sponge to spin/rotate within the belly button.

FIG. 2 shows an example of a personal hygiene device including a cleaning solution for cleaning a belly button. Each of the components are like their counterparts in FIG. 1 unless otherwise described herein. In this example, an interior of the device is shown. In some embodiments, the device 10 may further comprise a cleaning solution 16 releasably encapsulated within the handle 14, such that when the handle 14 is squeezed, the cleaning solution 16 is released from the handle 14 and through the conical sponge 12. The cleaning solution may be released through the sponge to a top of the sponge or may be released in various areas of the sponge such as in the helical grooves.

The device 10 of the present disclosure may be designed to be disposable. More specifically, the device 10 may be packaged in an individual sealed package, such that the device 10 may be easily disposed of after use thereof.

In various embodiments, the device 10 of the present disclosure may be made of any suitable material and may have any suitable or desired dimensions, wherein the size of the device may vary depending on the size of the belly button to be cleaned. In some embodiments, the conical sponge 12 may comprise any sponge suitable for personal hygiene purposes and the handle 14 may be made from, for example, plastic. The cleaning solution 16 may be any of various cleaning solutions 16 commonly used to clean a person, particularly pre-surgery or pre-medical procedure.

In various embodiments, the handle 14 may include a clear window to indicate how much solution is being used and/or remaining. Alternatively, the handle may be opaque.

To use the device of the present disclosure, a user may insert the tip of the conical sponge 12 into the belly button and twist the device using the handle 14, thus cleaning and removing matter from the belly button, resulting in a clean field. The device 10 may then be disposed of.

While the above describes that the device of the present disclosure may be used to clean the belly button, it is not limited to such use and, thus, may be used to clean other parts of the body or other objects.

Although the foregoing embodiments have been described in some detail for purposes of clarity of understanding, the invention is not limited to the details provided. There are many alternative ways of implementing the invention. The disclosed embodiments are illustrative and not restrictive.

What is claimed is:

1. A device, comprising:
a tapered sponge;
a handle coupled to and extending from a rear surface of the tapered sponge.

2. The device of claim 1, wherein the tapered sponge has a conical shape.

3. The device of claim 2, wherein the sponge includes at least one helical groove.

4. The device of claim 1, wherein the tapered sponge has an auger shape.

5. The device of claim 1, further comprising:
a cleaning solution releasably encapsulated within the handle.

6. The device of claim 1, wherein at least a portion of the handle is transparent.

7. The device of claim 1, wherein the handle is opaque.

8. The device of claim 1, wherein at least a portion of the tapered sponge is adapted to be placed into a belly button.

9. The device of claim 1, wherein the handle is adapted to be twisted to cause the tapered sponge to spin.

10. A method of manufacture, comprising:
providing a handle; and
attaching a tapered sponge to the handle.
